(19) United States
(12) Patent Application Publication
(10) Pub. No.: US 2004/0091963 A1
(43) Pub. Date: May 13, 2004

(54) Corrosion inhibitors

(76) Inventors: Hamilton Neil McMurray, Swansea (GB); David Anthony Worsley, Carmarthen (GB)

Correspondence Address: CONLEY ROSE, P.C., P. O. BOX 3267, HOUSTON, TX, 77253-3267, US

(21) Appl. No.: 10/343,077

(22) PCT Filed: Jul. 26, 2001
(86) PCT No.: PCT/GB01/03365

(30) Foreign Application Priority Data
Jul. 26, 2000 (GB) 0018164.4

(51) Int. Cl. C12P 21/06

(52) U.S. Cl. 435/69.1

(57) ABSTRACT

Use of a montmorillonite as a corrosion inhibitor for a metallic substrate. A corrosion inhibiting composition for a metallic substrate and a method of reducing corrosion on a metallic substrate. The montmorillonite preferably having a unit cell composition of [(Si₈)^{IV}.(Al_{3.33}Mg_{0.67})^{IV}.O₂₀(OH)₄]0.67M⁺ in which the superscripts (IV) and (VI) denote respective tetrahedral and octahedral layer cations and M⁺ denotes a univalent or equivalent compensating cationic charge.

The present invention is concerned with corrosion inhibitors for metallic substrates and in particular corrosion inhibitors comprising clay minerals.

Clay minerals chiefly comprise magnesium and aluminium silicates having layered lattice structures, the two most important examples of which are the minerals kaolinite and montmorillonite the respective principal components of the kaolin and bentonite clays.

Silicate minerals are derived from condensed forms of silicic acid, H₄SiO₄, in which each silicon atom is surrounded by four oxygen atoms which form the apices of a tetrahedral structure. These silicate tetrahedra are linked together in regular arrays by the sharing of common oxygen atoms to form chains like those of the pyroxine minerals, or are extended two-dimensional sheets or layers consisting of annellated cyclic groups of six silicate tetrahedra like those of the amphibole minerals. The tetrahedra in the silicate layers are usually oriented so that the three oxygens (the basal oxygens) of the tetrahedra lie on a common plane, with the fourth oxygen (the apical oxygen) lying on a second common plane.

Some metal hydroxides, notably those of aluminium, and magnesium, may also condense to form two-dimensional layered structures. Each layer of brucite, Mg(OH)₂, consists of a sheet of magnesium ions sandwiched between sheets of hydroxide ions. Each magnesium ion is surrounded by an octahedral arrangement of six hydroxide ions, while each hydroxide ion is shared by three magnesiums. Brucite has a trioctahedral structure, that is, a structure in which all octahedral sites are occupied by metal ions. Gibbsite, Al(OH)₃, layers have a similar structure but one-third of the octahedral sites are vacant. Gibbsite has a dioctahedral structure, that is, one in which only two-thirds of the octahedral sites are occupied by metal ions.

The dimensions of the above described silicate tetrahedral and metal hydroxide octahedral layers can be sufficiently matched to enable the two layers to condense to form a composite hydrated metal silicate layer in which the apical silicate oxygens replace a proportion of the octahedral hydroxyl groups. The tetrahedral and octahedral layers may be paired to form a 1:1 layer structure like that of kaolin, or two tetrahedral layers may flank each octahedral layer to form a 2:1 layer structure like that of the magnesium silicate, talc or the aluminium silicate, pyrophyllite. Various degrees of mismatch between the tetrahedral and octahedral layers are present in each mineral structure. This mismatch may be accommodated by distortion of the arrangements of tetrahedral and octahedral units within the structure and may also cause the morphology and chemistry of the mineral to differ from that of the parent species. The composite layers are described as trioctahedral or dioctahedral, depending on the structure of the octahedral layer component.

The layer silicate crystals are formed by the stacking of a number of these composite layers, often with intermediate layers of hydrated metal cations to compensate for charge discrepancies arising from structural defects in the silicate layers. Most of the silicate minerals used as fillers have crystal lattices formed from layered structures or from related structures containing arrays of abbreviated metal silicate sheets, or ribbons. Attapulgite is an example of a mineral containing a silicate ribbon lattice.

The hydrated metal silicate minerals generally used as fillers or pigments can be divided into several groups based on their crystalline structure. These groups are:

(i) Kaolinite and related 1:1-layer minerals, which include halloysite and one asbestos mineral, chrysotile;

(ii) Smectites and related 2:1-layer minerals, which include montmorillonite, hectorite, vermiculite, talc, the various chlorites, illites, and micas; and

(iii) The amphiboles, pyroxines, and related fibrous minerals having silicate ring, ribbon, or chain structures. These include wollastonite, attapulgite (palygorskite), sepiolite, and the remaining asbestos minerals

Kaolinite is a dioctahedral hydrated aluminosilicate containing alumina, silica, and water in a molecular ratio of 1:2:2. X-ray crystallography shows that the mineral has a 1:1-layer silicate structure. The unit cell of the kaolinite lattice has the composition [Si₂Al₂O₅(OH)₄]. The ideal kaolinite crystal consists of an array of hexagonal basal aluminosilicate layers, stacked like the pages of a book, the individual layers having an effective thickness or basal spacing of about 7.2 Å. They are bound together by hydrogen bonds between the hydroxylic Gibbsite-like surface of the octahedral layers and the oxygen sheet of the adjacent tetrahedral silicate layers.

Kaolins have a number of applications, including in particular for use as fillers in the plastics, paints and paper industries. AU-B-12527/83 also describes how inorganic oxides, such as Kaolinite, might be modified by ion-exchange to include cations of yttrium or one or more metals of the lanthanide group so as to be suitable as corrosion inhibitors. These corrosion inhibitors described in AU-B-12527/83 can be present in protective coatings based on film forming polymers or resins, such as paints, varnishes, lacquers and the like.

The smectites are a group of 2:1-layer minerals that includes the hydrated aluminium silicate, montmorillonite. An ideal montmorillonite may be defined as having the unit cell composition shown below, in which the subscripts (IV) and (VI) in the formula denote the respective tetrahedral and octahedral layer cations, and (M⁺) represents a univalent or equivalent compensating cationic charge

[(Si₈)^{IV}(Al_{3.33}Mg_{0.67})^{VI}.O₂₀(OH)₄]0.67M⁺

The isomorphous substitution consists predominantly of Mg-for-Al in the octahedral layer, resulting in a net anionic charge of 0.67 units per unit cell. The Mg-for-Al substitution has been shown to result in incomplete neutralisation of the negative charge on the apical oxygens and hydroxide groups coordinated to the magnesiums.

The anionic charge of the aluminosilicate layers of montmorillonites is neutralised by the intercalation of compensating cations and their coordinated water molecules. The montmorillonite crystal structure thus consists essentially of superimposed aluminosilicate layers, each of which is interleaved with a “layer” of hydrated, exchangeable compensating cations. These cations can alternatively be described as occupying the interlamellar spaces or the regions between the basal surfaces of opposing silicate layers. Although the compensating cations are normally located in the regions adjacent to the points of anionic charge on the basal surfaces, small anhydrous cations, principally Li⁺ or H⁺ ions, are capable of migrating through the basal surface oxygen sheet to the neighbourhood of the isomorphous substitution sites. The protons appear to associate with the octahedral hydroxyl groups, instead of forming hydroxyl groups by reacting with the incompletely neutralised oxygens.

Most native montmorillonites or bentonites have compositions close to that of the ideal mineral, but may contain additional octahedral isomorphous substitution, for example, octahedral Fe¹¹¹-for-Al, as well as some tetrahedral Al-for-Si substitution and other structural abnormalities. Na⁺, Ca²⁺, Mg²⁺, and Al³⁺ ions are the principal compensating cations in the natural bentonites, the dominant ion depending on the origin of the particular deposit. These cations may be exchanged with other ions, either during weathering of the deposit or during processing of the clay.

Wyoming bentonite, which is believed to have been formed by the weathering of volcanic ash in an ancient sea, with little subsequent leaching, has Na⁺ ions as the principal compensating cations. The cation exchange capacities of montmorillonites from this source are about 0.8 to 1.0 mequiv/g. The Texas bentonites have a slightly different genesis, and their constituent montmorillonites have a higher cation exchange capacity and a greater degree of tetrahedral isomorphous substitution than the Wyoming types. As a result of weathering and natural ion exchange, the Texas bentonites contain hydrated Ca²⁺ ions as the compensating cations.

Bentonites and their derivatives can generally be divided into four categories: the swelling (Na⁺) bentonites, for example, the natural Wyoming mineral or ion-exchanged bentonites from other sources; the non-swelling (predominantly Ca²⁺) bentonites, which are the most widely distributed natural form; the organophilic bentonites; and the acid bentonites or bleaching clays, often used as catalysts and as decolourising agents in the treatment of vegetable and mineral oils.

The hitherto major applications for the bentonites are as additives in the iron ore pelletising, metal foundry, and oil-drilling industries. For many of these uses, the bentonite requires processing, typically drying the raw clay, which may contain 40% water, pulverising the dry product (15% water), and classification of the powdered clay in air cyclones. The non-swelling bentonites may be converted to the swelling sodium form, for example, by treatment with sodium carbonate or other salts that insolubilise the Ca²⁺ present in the raw clay.

We have now found a new use for montmorillonites and more particularly we have found that montmorillonites are useful as corrosion inhibitors, particularly corrosion inhibitors for metallic substrates, such as galvanised steel and the like.

organically coated galvanised steels (OCS) have been of increasing importance as architectural cladding in the construction industry. The principal mode of failure experienced by these materials has been corrosion occurring at exposed metallic cut edges. The nature of this failure has been shown to involve a combination of coating delamination due to anodic metal dissolution (reaction 1) and physical degradation of coatings brought about by increased pH as a result of localised cathodic oxygen reduction (reaction 2):

Zn₍ₛ₎→Zn²⁺_{(aq)}+2e⁻ (1)

O_{2(g)}+2H₂O₍ₗ₎+4e⁻→4OH⁻_{(ag)} (2)

It has been possible to slow the rate of corrosion failure by inclusion of anodic and cathodic corrosion inhibitors in the organic coating systems to lower the rate of reactions (1) and (2) respectively. Sparingly soluble salts based on chromate (Cr(VI)) have been used extensively in inhibitor pigments for this purpose. CrO₄²⁻ is often regarded as an anodic precipitation inhibitor but there has also been evidence that, in its reduced form, Cr³⁺, it may slow the cathodic process. However, despite the efficiency of chromate as an inhibitor there has been increasing pressure to develop effective alternative inhibitor systems due to its known toxic and carcinogenic properties.

The present invention alleviates the problems hitherto experienced with known corrosion inhibitors for metallic substrates and there is provided by the present invention use of a montmorillonite as a corrosion inhibitor for a metallic substrate.

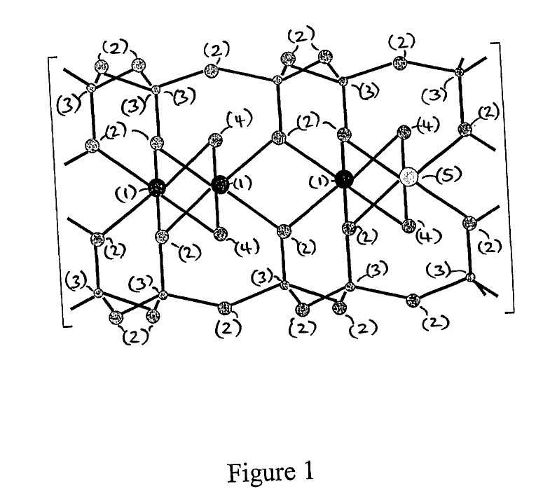

Substantially as hereinbefore described a montmorillonite can be defined as having a unit cell composition of

[(Si₈)^{IV}.(Al_{3.33}Mg_{0.67})^{VI}.O₂₀(OH)₄)]0.67M⁺

in which the superscripts (IV) and (VI) denote respective tetrahedral and octahedral layer cations and M⁺ denotes a univalent or equivalent compensating cationic charge. Preferably a montmorillonite used according to the present invention has a unit cell substantially as defined above and substantially as further illustrated in FIG. 1.

Preferably the compensating cationic charge present in a montmorillonite used according to the present invention comprises one or more compensating cations selected from alkali metal cations, alkali earth metal cations, yttrium and lanthanide cations. Typically the compensating cationic charge may comprise one or more compensating cations selected from the group consisting of calcium, barium, strontium, yttrium, cerium and lanthanum. According to a first aspect of the present invention the compensating cationic charge present in the montmorillonite may predominantly comprise calcium. According to a second aspect of the present invention the compensating cationic charge may predominantly comprise cerium. In a particularly preferred aspect of the present invention the compensating cationic charge predominantly comprises calcium and the montmorillonite used according to the present invention is a naturally occurring montmorillonite obtained from Texas bentonite substantially as hereinafter described.

More particularly, the present invention is preferably concerned with the use, as a corrosion inhibitor for a metallic substrate, of a montmorillonite obtainable from naturally occurring bentonite clay. Suitable such naturally occurring bentonite clays include Texas bentonite, Wyoming bentonite and the like. A preferred montmorillonite for use according to the present invention is obtainable from or in the form of Texas bentonite. Typically in the case where the montmorillonite is obtainable from Texas bentonite, the compensating cationic charge of the montmorillonite predominantly comprises calcium substantially as hereinbefore described.

Preferably according to the present invention there is provided use, as a corrosion inhibitor for a metallic substrate, of a naturally occurring montmorillonite. It is particularly preferred that there is provided by the present invention use, as a corrosion inhibitor for a metallic substrate, of a naturally occurring montmorillonite obtained from Texas bentonite.

It may be alternatively preferred, however, that a montmorillonite for use according to the present invention includes compensating cations that have been introduced by ion exchange to replace compensating cations originally present in the naturally occurring montmorillonite. Typically, for example, the compensating cationic charge of ion exchanged montmorillonite for use according to the present invention may predominantly comprise one or more cations selected from the group consisting of cerium (preferably), yttrium, lanthanum, barium and strontium.

Typically there is still further provided by the present invention a corrosion inhibiting composition for a metallic substrate, which composition comprises a corrosion inhibiting amount of a montmorillonite substantially as hereinbefore described together with a carrier medium therefor. Typically the carrier medium may be selected from the group consisting of polyester resins, epoxy resins, polyacetals (such as polyvinyl butyral or the like), alkyd (polyester) resins and the like. Other suitable ingredients present in a composition according to the present invention may comprise suspension agents, extenders (such as silica and the like)), flow agents (typically acrylic), cross linkers (typically amines) and any other further components of the type suitable for inclusion in corrosion inhibiting compositions as provided by the present invention and which are compatible with a montmorillonite as employed according to the present invention.

Typically a corrosion inhibiting composition according to the present invention is suitable for application as a protective coating for a metallic substrate. The protective coating may typically be in the form of a paint, varnish, lacquer or the like for the metallic substrate.

A montmorillonite for use substantially as hereinbefore described may typically be present in a corrosion inhibiting composition according to the present invention as a filler therefor and may be included in an amount in the range of 10 to 30% by volume (typically 15 to 25% by volume, such as about 19% by volume), based on the substantially cured composition when applied to a metallic substrate.

Preferably the properties of a montmorillonite for use substantially as hereinbefore described in a corrosion inhibiting composition according to the present invention should be such that the montmorillonite should remain in suspension in the composition prior to application to a metallic substrate and should not substantially affect either the ease of application of the coating or the desired resulting properties (such as smoothness and the like) of the applied coating. Typically the particle size of the montmorillonite should be selected to achieve the above desired effects. Particularly, montmorillonite particles employed in the present invention should have a maximum dimension of less than about 20 μm.

Metallic substrates to be protected from corrosion by the use of a montmorillonite according to the present invention typically include steel artefacts (particularly sheet steel or galvanised steel), aluminium alloy artefacts and the like. There is typically further provided by the present invention a metallic substrate to which is applied a corrosion inhibiting amount of a montmorillonite substantially as hereinbefore described. More particularly, there is further provided by the present invention a metallic substrate provided with a corrosion inhibiting composition substantially as hereinbefore described.

The present invention also provides a method of alleviating the corrosion of a metallic substrate, which method comprises applying to the substrate a corrosion inhibiting amount of a montmorillonite substantially as hereinbefore described (which montmorillonite is preferably present in a corrosion inhibiting composition substantially as hereinbefore described).

The present invention will now be further illustrated by the following figures which do not limited the scope of the invention in any way.

FIG. 1 is a unit cell of a montmorillonite for use according to the present invention; and

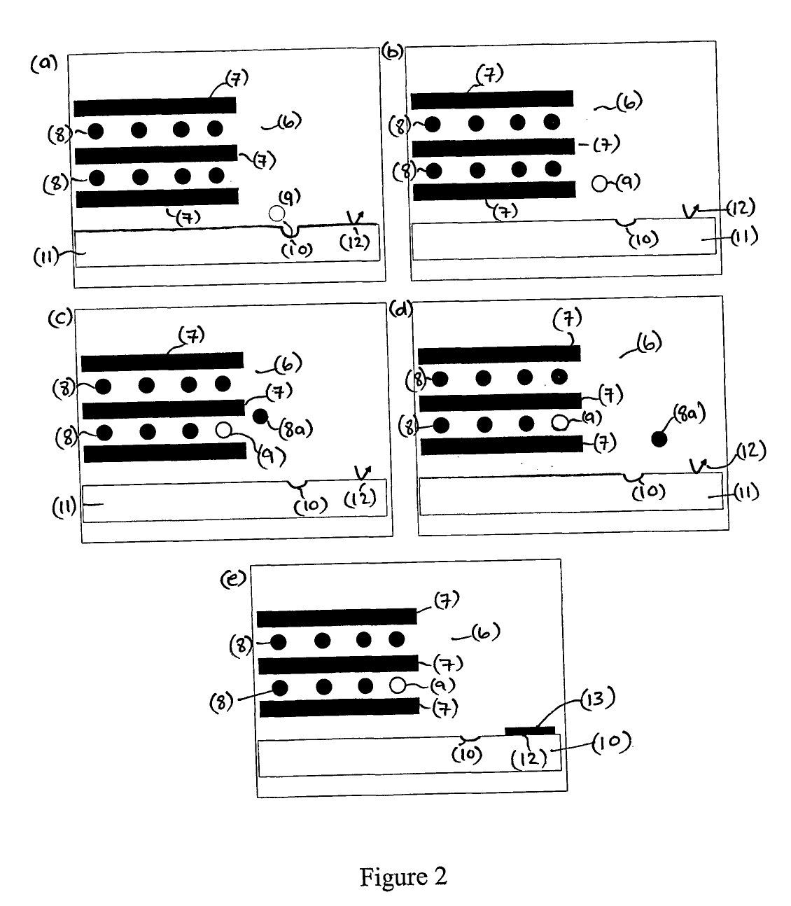

FIG. 2 is a schematic representation illustrating the mechanism of corrosion inhibition achieved by the present invention.

Referring to FIG. 1, (1) represent aluminium atoms, (2) represent oxygen atoms, (3) represent silicon atoms, (4) represent hydroxide molecules and (5) represents a magnesium atom. The unit cell shown in FIG. 1 has a net anionic charge of 0.67 units and this is substantially neutralised by the intercalation of compensating cationic charge in the overall montmorillonite crystal lattice. The overall montmorillonite crystal lattice comprises superimposed aluminosilicate layers, each of which is interleaved with hydrated compensating cations substantially as hereinbefore described and as further schematically illustrated hereinafter by reference to FIG. 2.

Referring to FIG. 2, montmorillonite (6) comprises negatively charged aluminosilicate layers (7) and compensating cations (8). Zn²⁺ ions (9) are liberated at an anodic site (10) of zinc coated steel substrate (11). At cathodic site (12) the following hydroxyl liberating reaction (substantially as hereinbefore described) occurs:

O₂+2H₂O+4e⁻→4OH^{'}

As shown in step (a), corrosion of the surface of steel substrate (11) is initiated by liberation of Zn²⁺ ion (9) at anodic site (10). The above described hydroxyl liberating reaction occurs at cathodic site (12).

As shown in step (b), liberated Zn²⁺ ion (9) diffuses in solution towards montmorillonite (6).

As shown in step (c), liberated Zn²⁺ ion (9) undergoes ion exchange with a compensating cation (8a), Zn²⁺ ion (9) is thus immobilised in montmorillonite structure (6).

As shown in step (d), compensating cation (8a) diffuses towards cathodic site (12) of steel substrate (11).

As shown in step (e), a protective layer (13) is formed at cathodic site (12) of substrate (11). Protective layer (13) is formed by compensating cation (8a) reacting with hydroxyl ions present at cathodic site (12). Compensating cation (8a) thus acts as an inhibitor ion, lowering the level of cathodic activity. Although not illustrated by FIG. 2, further inhibitor ions, such as protons or the like, could be released as a result of metal ion hydrolysis of substrate (11) underlying montmorillonite (6).

The present invention will now be still further illustrated by the following Example, which does not limit the scope of the invention in any way.

### EXAMPLE 1

A naturally occurring, montmorillonite clay, Wyoming bentonite was used. Substantially as hereinbefore described Wyoming bentonite is an alumino-silicate material comprising negatively charged layers (resulting from isomorphic substitution of aluminium ions in the structure of the matrix by magnesium), held together by electrostatic attraction, with the compensating cations (predominantly Na⁺) necessary for charge neutrality residing between them. The ion exchange capacity of GG Wyoming bentonite is 0.70 mequiv/g, i.e. every gram will exchange 0.7 millimole of univalent cations, 0.35 millimole of divalent cations and so forth.

A Ce³⁺ exchanged Wyoming bentonite pigment was prepared by replacing the naturally occurring Ca²⁺ ions in the Wyoming clay matrix with Ce³⁺, following a standard exchange procedure as described in J. M. Adams et al, Journal of Catalysis, 58, 1979, 238. 200 g quantities of the “raw” Wyoming bentonite were stirred in 1 litre volumes of 1 mol dm⁻³ solution of CeCl₃ (99.9% purity, Aldrich) in distilled water, for 24 hours. The pigment was then separated from the liquor using a Hertz centrifuge at 2000 rpm. The matrices were then washed in fresh distilled water until all residual non-exchanged ions were removed, before undergoing a final wash with ethanol to remove water. The resulting pigment was dried in an oven at 70° C. for four hours. Pigment preparation was completed by grinding and milling of the dried powders to attain a suitable particle size (<20 μm diameter) for inclusion in primer formulations.

The composition of the Wyoming bentonite prior to and following ion exchange was investigated by acid digestion of the inorganic matrix in aqua regia followed by inductively coupled plasma/mass spectrometry analysis. This indicated the level of exchangeable sodium and calcium cations present in the naturally occurring Wyoming to be 10700 and 7000 mg/kg respectively. Following ion exchange these cations were seen to have been replaced with 31500 mg/kg Ce³⁺, 4700 mg/kg of Ca²⁺ remained unexchanged.

The Ce³⁺ exchanged Wyoming bentonite pigment was incorporated into a polyester resin based primer system. An identical primer was prepared with the non-exchanged bentonite in order to also demonstrate the efficiency of the naturally occurring Ca²⁺ ion exchange materials. In order to attain an indication of relative performance two standard systems incorporating (I) a sparingly soluble strontium chromate salt pigment and (ii) the commercial chromate-free alternative, calcium exchanged silica (available under the trade mark Sheildex—grade CP4) were also prepared in an identical resin. In each case the active pigment loading was 38% of the total weight of non-solvent ingredients. Pigments were fully dispersed into the polyester resin by the use of a Dispermat high shear mixer followed by running through a 50 ml Eiger horizontal bead mini mill, until a final pigment particle size of sub 5/m diameter (measured using a Hegman draw down gauge) was achieved.

The anti-corrosion performance of the primer systems was assessed by salt spray testing, according to ASTM B119/97.

Test panels were prepared using 0.52 mm gauge sheet steel which had been hot-dip coated on both sides to give a 20 μm zinc layer (composition Zn 99.85%, Al 0.15% with minimal formation of iron-zinc intermetallic phases) as the substrate, obtained from British Steel Strip Products.

Prior to organic coating application the substrate was firstly pre-treated by Henkel Metal Chemicals to provide a good chemical and physical key for the subsequent coating layers. Primers systems were applied to give a 5 μm dry film thickness following curing at a peak metal temperature (PMT) of 216° C. in a Math is Lab dryer. Architectural polyester (available from PPG Industries) was then applied over the primers to 18 μm dry film thickness, giving a total organic layer thickness of 23 μm. In each case the coatings were applied in the laboratory by the draw down method using gauged wire wound coating bars (available from Sheen Instruments). Organic coating thickness measurements of the cured films were made by magnetic induction using a Fischer Permascope.

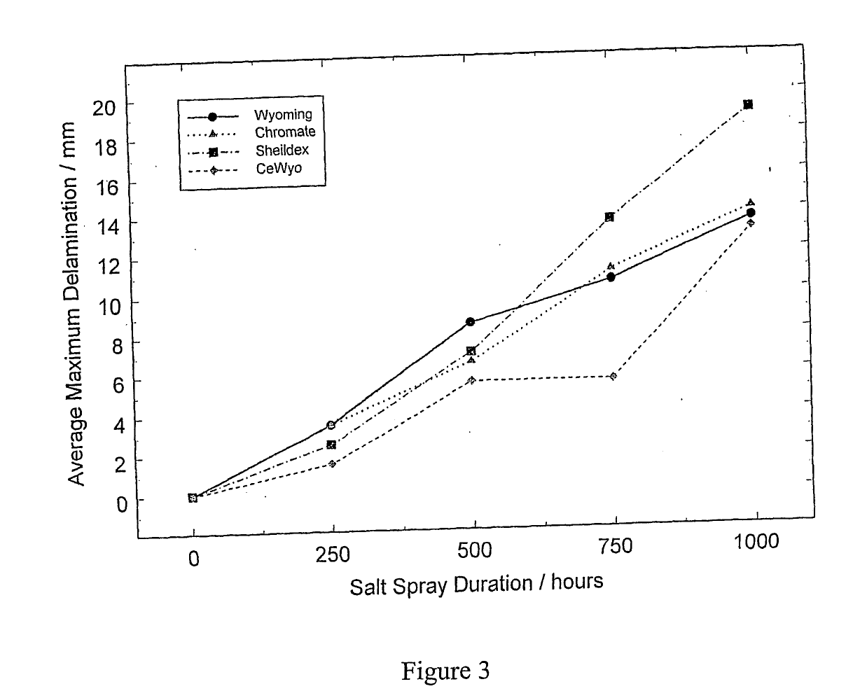

Test panels, produced in duplicate, were then subjected to 1000 hours salt spray testing with observations recorded every 250 hours. The results are presented in FIG. 3 as the average of the maximum coating delamination observed from the cut edge on the duplicate panels with respect to the salt spray exposure time and where

(a1) represents the results for Wyoming bentonite;

(b1) represents the results for commercially available strontium chromate;

(c1) represents the results for commercially available calcium exchanged silica; and

(d1) represents the results for cerium exchanged Wyoming bentonite.

The results show that the above described Ce³⁺ exchanged Wyoming pigment containing primer system offered superior cut edge corrosion performance to both the tested commercial systems throughout the test period. It can also be seen that the primer containing the naturally occurring Wyoming clay also produced a similarly high level of corrosion performance.

The above results clearly illustrate that naturally occurring calcium exchanged bentonite clays offer equivalent performance to commercially available SrCrO₄ with greater performance than current commercial calcium based ion exchange materials.

### EXAMPLE 2

A naturally occurring, montmorillonite clay, Wyoming bentonite was used. The Wyoming bentonite was substantially as hereinbefore described.

A Ce³⁺ exchanged Wyoming bentonite pigment was prepared by replacing the naturally occurring Ca²⁺ ions in the Wyoming clay matrix with Ce³⁺, using the same method described with reference to Example 1.

The Ce³⁺ exchanged Wyoming bentonite pigment was incorporated into an epoxy based primer system, again using the same method as set out in Example 1. The resultant composition was applied to a mild steel surface as a corrosion inhibitor.

### EXAMPLE 3

A naturally occurring, montmorillonite clay, Wyoming bentonite was used. The Wyoming bentonite was substantially as hereinbefore described.

A Ce³⁺ exchanged Wyoming bentonite pigment was prepared by replacing the naturally occurring Ca²⁺ ions in the Wyoming clay matrix with Ce³⁺, using the same method described with reference to Example 1.

The Ce³⁺ exchanged Wyoming bentonite pigment was incorporated into a polyurethane primer system using the method identified in Example 1. The resultant composition was applied to an aluminium surface as a corrosion inhibitor.

### EXAMPLE 4

A naturally occurring montmorillonite clay was exhaustively exchanged with a mixture of trivalent rare earth cations in the ratio in which they occur naturally in the mineral Monazite. Exchange was effected by repeated dispersion of the montmorillonite in mixed aqueous solutions of the rare earth chloride salts.

Monazite is a naturally occurring ore for the trivalent lanthenide ions (cerium, lanthenum etc) which are proposed as exchangeable ions within the montmorillonite matrix. Monazite occurs naturally as a heavy dark sand which is essentially a lanthanide orthophosphate. The distribution of elements is usually such that La, Ce, Pr and Nd make up about 90% with Y and other heavier elements making up the remainder. The ore is acid digested to release the trivalent ions into solution.

The exhaustively exchanged material was then washed, dried and ground as detailed in Example 1. The ground pigment was then dispersed in a polyacetal primer formulation in a manner as outlined in Example 1, and applied to mild steel which had been hot-dip galvanised with aluminium (5%) and zinc (95%) alloy coating.

1. Use of montmorillonite as a corrosion inhibitor for a metallic substrate.

2. Use according to claim 1, characterised in that the montmorillonite has a unit cell composition of
[(Si₈)^{IV}.(Al_{3.33}Mg_{0.67})^{VI}.O₂₀(OH)₄]0.67M⁺
in which the superscripts (IV) and (VI) denote respective tetrahedral and octahedral layer cations and M+denotes a univalent or equivalent compensating cationic charge.

3. Use according to claim 2, characterised in that the compensating cationic charge present in the montmorillonite comprises one or more compensating cations selected from alkali metal cations, alkali earth metal cations, yttrium and lanthanide cations.

4. Use according to claim 2 or 3, characterised in that the compensating cationic charge comprises one or more compensating cations selected from the group consisting of calcium, barium, strontium, yttrium, cerium and lanthanum.

5. Use according to any of claims 2 to 4, characterised in that the compensating cationic charge predominantly comprises calcium or cerium.

6. Use according to any of claims 1 to 5, characterised in that the montmorillonite is obtainable from naturally occurring bentonite clay.

7. Use according to claim 6 characterised in that the naturally occurring bentonite clay includes Texas bentonite and/or Wyoming bentonite.

8. Use according to any of claims 2 to 7, characterised in that the compensating cationic charge comprises calcium and the montmorillonite is a naturally occurring montmorillonite obtained from Texas bentonite.

9. Use, as a corrosion inhibitor for a metallic substrate, a naturally occurring montmorillonite.

10. Use according to claim 9 of a naturally occurring montmorillonite in the form of Texas bentonite.

11. Use according to any preceding claim, characterised in that the montmorillonite includes compensating cations that have been introduced by ion exchange to replace compensating cations originally present in the naturally occurring montmorillonite.

12. Use according to claim 11, characterised in that the compensating cationic charge of ion exchanged montmorillonite comprises one or more cations selected from the group consisting of cerium (which is preferred), yttrium, lanthanum, barium or strontium.

13. A corrosion inhibiting composition for a metallic substrate, which composition comprises a corrosion inhibiting amount of a montmorillonite in a film forming carrier medium.

14. A corrosion inhibiting composition according to claim 13, characterised in that the montmorillonite has a unit cell composition of
[(Si₈)^{IV}.(Al_{3.33}Mg_{0.67})^{VI}.O₂₀(OH)₄]0.67M⁺
in which the superscripts (IV) and (VI) denote respective tetrahedral and octahedral layer cations and M⁺ denotes a univalent or equivalent compensating cationic charge.

15. A corrosion inhibiting composition according to claim 13 or 14, characterised in that the film forming carrier medium is selected from the group consisting of polyester resins, epoxy resins, polyacetals (such as polyvinyl butyral), alkyd (polyester) resins and polyurethane.

16. A corrosion inhibiting composition according to any of claims 13 to 15, characterised in that the composition further includes suspension agents, extenders (such as silica), flow agents (typically acrylic) and/or cross linkers (typically amines).

17. A corrosion inhibiting composition according to any of claims 13 to 16 which is arranged to be applied as a protective coating (typically in the form of a paint, varnish, lacquer or the like) for the metallic substrate.

18. A corrosion inhibiting composition according to any of claims 13 to 17, characterised in that the montmorillonite is included in the composition in an amount in the range 10 to 30% by volume (typically 15 to 25% by volume, such as about 19% by volume), based on the composition when applied to a metallic substrate (which is typically substantially cured).

19. A corrosion inhibiting composition according to any of claims 13 to 18, characterised in that the montmorillonite is in suspension in the composition prior to application to the metallic substrate.

20. A corrosion inhibiting composition according to any of claims 13 to 19, characterised in that the montmorillonite has a maximum particle size of less than about 20 μm.

21. A corrosion inhibiting composition according to any of claims 13 to 20, characterised in that the substrate includes steel artefacts (such as sheet steel or galvanised steel) or aluminium artefacts.

22. A metallic substrate having coated thereon, a corrosion inhibiting amount of montmorillonite.

23. A metallic substrate according to claim 22, characterised in that the montmorillonite is in a film forming carrier medium.

24. A substrate according to claim 22 or 23, characterised in that the montmorillonite has a unit cell composition of
[(Si₈)^{IV}.(Al_{3.33}Mg_{0.67})^{VI}.O₂₀(OH)₄]10.67M⁺
in which the superscripts (IV) and (VI) denote respective tetrahedral and octahedral layer cations and M⁺ denotes a univalent or equivalent compensating cationic charge.

25. A method of reducing corrosion of a metal substrate, which method includes applying to the substrate a corrosion inhibiting amount of montmorillonite.

26. A method according to claim 25, characterised in that the montmorillonite has a unit cell composition of
[(Si₈)^{IV}.(Al_{3.33}Mg_{0.67})^{VI}.O₂₀ (OH)₄]0.67M⁺
in which the superscripts (IV) and (VI) denote respective tetrahedral and octahedral layer cations and M+denotes a univalent or equivalent compensating cationic charge.

27. A method according to claim 25 or 26, characterised in that the montmorillonite is applied as a corrosion inhibiting composition according to any of claims 14 to 22.
